(19) United States
(12) Patent Application Publication
(10) Pub. No.: US 2025/0156677 A1
(43) Pub. Date: May 15, 2025

(54) MACHINE LEARNING-BASED AUTOMATED ABNORMALITY DETECTION IN MEDICAL IMAGES AND PRESENTATION THEREOF

(71) Applicant: Arterys Inc., San Francisco, CA (US)

(72) Inventors: Matthew Joseph DiDonato, Redwood City, CA (US); Daniel Irving Golden, Palo Alto, CA (US); John Axerio-Cilies, Berkeley, CA (US); Taryn Nicole Heilman, Arvada, CO (US)

(21) Appl. No.: 19/026,072

(22) Filed: Jan. 16, 2025

Related U.S. Application Data
(63) Continuation of application No. 17/285,731, filed on Apr. 15, 2021.
(60) Provisional application No. 62/770,038, filed on Nov. 20, 2018.

(51) Int. Cl. G06N 3/02; G06N 7/01; G16H 30/40; G16H 50/20

(52) U.S. Cl. CPC G06N 3/02; G06N 7/01; G16H 30/40; G16H 50/20

(57) ABSTRACT

The presently disclosed technology relates to medical image processing. An example method includes receiving medical image data which represents an anatomical structure and processing the received image data through convolutional neural network (CNN) to generate predictions. The predictions can include abnormality location proposals and abnormality class probabilities associated with each abnormality location proposals.

### BACKGROUND

### Technical Field

The presently disclosed technology is generally related to medical image processing including abnormality detection, characterization, and visualization.

### Description of the Related Art

Typically, medical imaging is the technique and process of creating visual representations of the interior of a body for clinical analysis and medical intervention, as well as visual representation of the function of some organs or tissues (physiology). One of the goals of medical imaging is to reveal internal structures hidden by the skin and bones, as well as to diagnose and treat disease.

### BRIEF DESCRIPTION OF THE DRAWINGS

FIG. 1 shows an example flow diagram of one implementation of an Abnormality Flagging User Interface.

FIG. 2 shows an example user interface for one implementation of a mammography flagging system.

FIG. 3 is an example flow diagram describing how scan abnormality characteristics may be indicated.

FIG. 4 shows an example flow diagram of one implementation of the Abnormality Detection User Interface.

FIG. 5 shows an example user interface for one implementation of a mammography abnormality detection system with a rectangular bounding box indication.

FIG. 6 shows an example user interface for one implementation of a mammography abnormality detection system with a segmentation contour indication.

FIG. 7 shows an example user interface for one implementation of a mammography abnormality detection system with a segmentation mask indication.

FIG. 8 shows an example user interface for one implementation of a mammography abnormality detection system with a point indication.

FIG. 9 shows an example system diagram of one implementation of the Abnormality Detection Model Inference with a single CNN model.

FIG. 10 shows an example system diagram of one implementation of the Abnormality Detection Model Inference with separate CNN models for detection and classification.

FIG. 11 shows an example system diagram of one implementation of a whole study classification system.

FIG. 12 shows an example processor-based device.

### DETAILED DESCRIPTION

### Overview

Described herein is a system that can be used to perform any of several functions on medical images:
Detection of abnormalities
Characterization of abnormalities.
Display of detected abnormalities and their characteristics
Display of multiple images or studies in a worklist along with an indication of known or suspected abnormalities to facilitate timely reading of the most time sensitive studies,
Any combination of the above

At least one embodiment is designed for use in the context of mammography screening exams. In this embodiment, the abnormalities of interest are primarily those that influence the likelihood of a diagnosis of cancer, as described in the Breast Imaging Reporting and Data System (BI-RADS) published by the American College of Radiology (ACR). See ACR BI-RADS Atlas® 5th Edition, available at https://www.acr.org/Clinical-Resources/Reporting-and-Data-Systems/Bi-Rads. These abnormalities may include suspected malignant lesions as well as optionally suspected benign lesions, such as fibroadenomas, cysts, lipomas, and others.

Beyond breast imaging, many other embodiments of the described system involving different types of abnormalities observed in radiology are possible, including but not limited to:
Solid cancers of other organs, including but not limited to brain, lung, liver, bone, and others,
Traumatic injuries, such as cerebral hemorrhage, bone fractures, and others,
Ischemia or vascular stenosis,
Multiple sclerosis and other non-malignant lesions,
Any combination of the above

We describe separately embodiments of each of these systems:
Abnormality Flagging User Interface
Abnormality Detection User Interface
Abnormality Detection Machine Learning Model Inference

### Abnormality Flagging User Interface

At least one purpose of the Abnormality Flagging User Interface is to provide a list of studies to a radiologist and call their attention to those studies that may be higher priority than others. Those studies may be of higher priority for many reasons, including but not limited to:
They need to be read by a radiologist,
They need to be read by a radiologist urgently,
They are likely to contain abnormalities,
Any combination of the above

In radiological terms, multiple images that make up a single acquisition are defined as a series. Multiple series that correspond to a single scanning session are defined as a study. A study may contain one or more series, and a series may contain one or more images. These precise definitions are not integral to the design of the system described herein; for the purpose of this description, a study is defined as a collection of one or more related images of a single patient from a single scanning session.

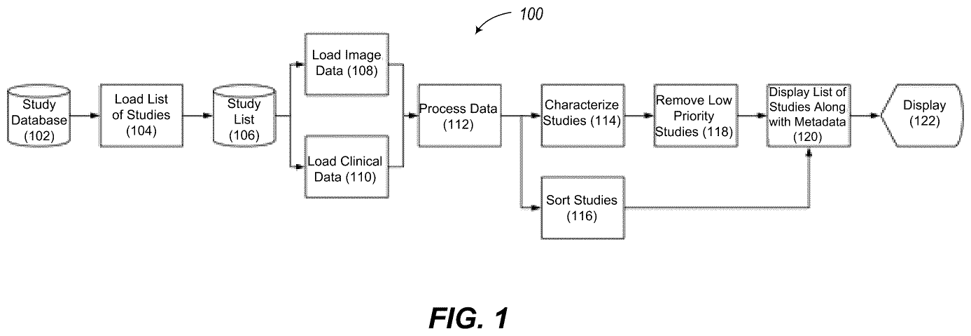

FIG. 1 shows an example flow diagram (100) of one embodiment of the operation of the Abnormality Flagging User Interface. In this embodiment, images that compose a study are originally contained in a Study Database (102). This database may contain one or more studies. In embodiments that involve mammography screening, the database may contain only mammography studies, or it may contain additional types of studies. The system loads a list of studies at (104), resulting in a study list at (106). This list of studies is a subset of the studies in the study database and may be filtered based on any of several different criteria, including, but not limited to:
Studies that have not yet been read by a radiologist at all,
Studies that have been read by a junior radiologist and require a confirmatory read by a senior radiologist,
Studies that were acquired recently (e.g., the past week, month, or year),
Studies for a particular patient,
Studies that were acquired for a particular indication, such as mammography,
Studies of a particular modality, such as mammography,
Any combination of the above

For each of the studies in the list, image (pixel) data is loaded at (108). That image data is optionally combined with clinical data (110) or other data and processed at (112). The processing may include inference by a convolutional neural network (CNN), inference by other machine learning algorithms, heuristic algorithms, decision trees, other image processing, any combination of the above or other processing techniques. Optionally, if the processing results in a sortable characteristic (such as percent likelihood of an abnormality being present), the images may be sorted by that characteristic at (116). Optionally, one or more characteristics may be associated with each study at (114), without those characteristics necessarily being used to sort or rank the studies. In the case of screening mammography, various characteristics may be assessed, including but not limited to:
Breast density,
Likelihood of an abnormality,
Likelihood of a malignant abnormality,
Image acquisition quality,
Any combination of above

If characteristics of the studies are determined, some studies may optionally be removed from the list at (118), such as those that have a low likelihood of containing an abnormality, or those that have a low likelihood of diagnostic quality. The list of studies, which in some embodiments will be curated or sorted, is then displayed to the user at (120) on a display at (122). The displayed list of studies may include some indication of the characterization of the studies, such as a “high priority” flag adjacent to studies characterized as likely to contain one or more abnormalities. Multiple indications may be used for separate abnormalities or groups of abnormalities, such as, e.g., a separate indication for any of, but not limited to:
mass-like abnormalities,
calcification abnormalities,
asymmetry abnormalities,
abnormalities that require additional procedures to diagnose (e.g., an ambiguous lesion that requires biopsy for diagnosis),
abnormalities that require additional procedures to treat (e.g., a likely malignant lesion),
others,
any combination of the above

The data processing at (112) can be accomplished in various ways, including by one or more convolutional neural network (CNN) models. One or more CNNs may be a detection model, and one or more may be a segmentation model. One or more CNNs may return any of various results, including but not limited to:
The likelihood of one or more abnormalities in the image data,
The locations of one or more abnormalities in the image data
The probability of one or more entire images containing an abnormality,
The probability of one or more anatomical organs containing an abnormality (such as one or both breasts, or one or both lungs)

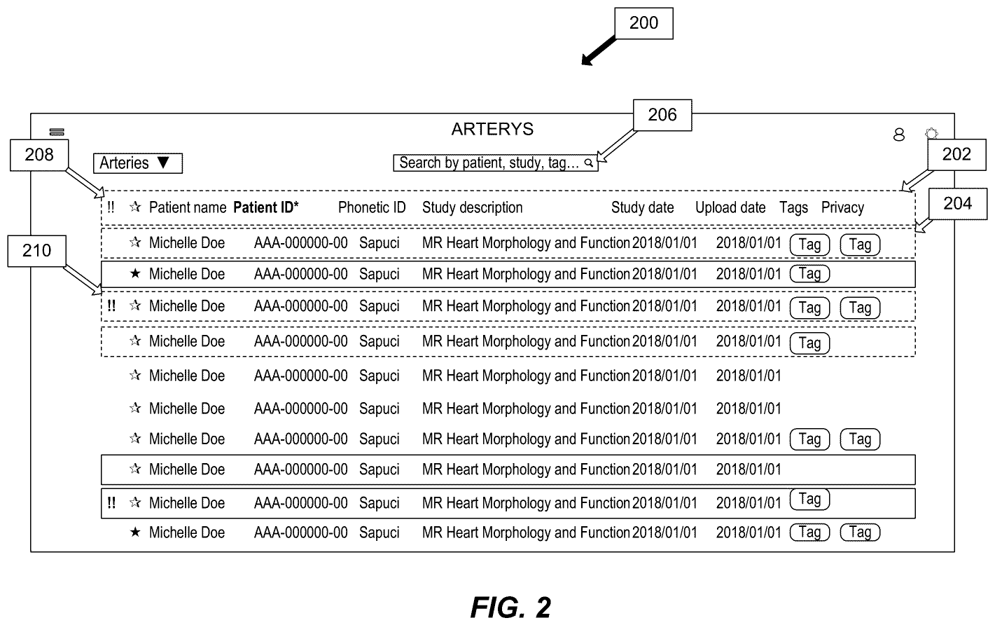

FIG. 2 shows one embodiment of a user interface (200) for the displaying the list of studies and metadata in (120). The header of the list at (202) defines the columns and each row in the list at (204) is a separate study. The user can search for studies or otherwise filter them using the search bar at (206). In this embodiment, the study characterization assessed at (114) is a Boolean indicator that the given study is likely to contain an abnormality; the indicator is a pair of exclamation marks (!!) (208). A study indicated with the exclamation marks is said to be “flagged.” An example study that has been flagged is shown at (210). In this embodiment, the studies in the list are not sorted by probability of containing an abnormality but are instead sorted by, for example, date or time of acquisition.

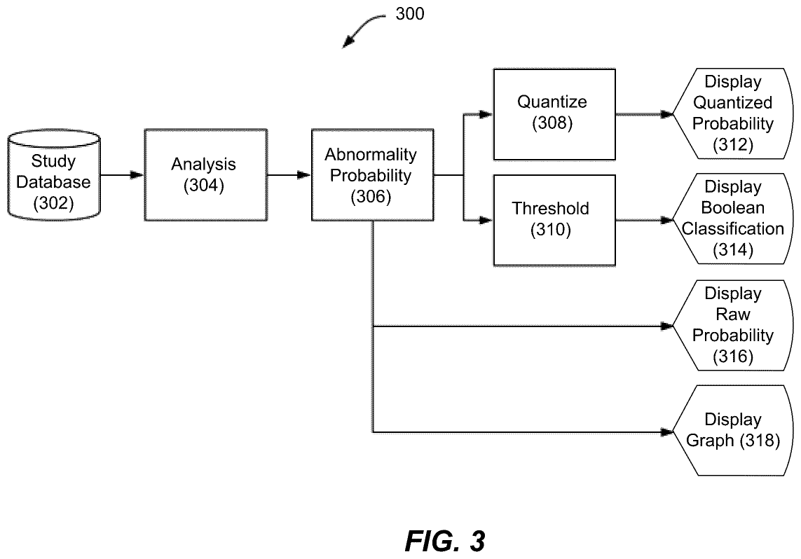

FIG. 3 shows an example flow diagram (300) of one embodiment of the operation of a system for determining how abnormality-related characteristics of a study may be calculated or shown. Many different abnormality-related characteristics may be displayed, including but not limited to:
Likelihood of a study containing any abnormality
Likelihood of a study containing any of a subset of abnormalities
Likelihood of a study containing an abnormality that is suspicious for cancer
Likelihood of a study containing an abnormality that is suspicious for a specific subtype of cancer
Any combination of above

In this embodiment, images that compose a study are originally contained in a Study Database (302). Those studies are analyzed at (304). In this embodiment, the results of the analysis are an abnormality probability at (306). This probability itself may be displayed any of several ways, including as a raw probability value, such as a percentage at (316), or as a graph, color scale, or other visual indicator of the percentage at (318). The abnormality probability may be quantized at (308) into discrete risk levels and that risk level may be displayed as a textual label, such as “low,” “medium” and “high,” or using a visual indicator such as a number of bars or dots, or a color, such as green, yellow and red, at (312). Alternatively, the abnormality probability may be thresholded into a Boolean True or False value at (310) which would indicate whether the abnormality is likely to be present; this could be displayed as a flag, as in (210), as a highlight, or via other indicators at (314).

### Abnormality Detection User Interface

One purpose of the Abnormality Detection User Interface is to provide a visual indication of the location of suspected abnormalities on the original radiological pixel data. These visual indications guide the user's eye to the abnormality to allow the user to confirm or deny any of the presence, characteristics, or diagnosis of the abnormality. Some embodiments of this type of interface may be referred to as Computer Aided Detection, or CAD or CADe.

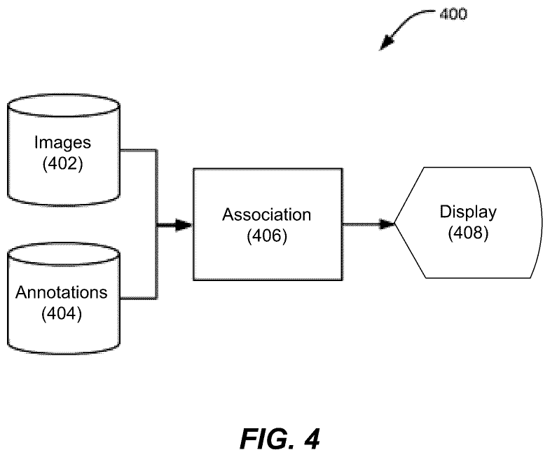

FIG. 4 shows an example flow diagram (400) of one embodiment of the operation of a detection user interface under the presumption that annotations that indicate the locations and optionally the characteristics of abnormalities have already been collected, either manually or automatically. In this embodiment, images (optionally arranged in collections of studies) are contained in a database at (402). Annotations associated with those images, which may include bounding boxes, segmentations, points, or other pixel references, are also contained in a database at (404). The system associates the annotations with their respective images at (406) and displays the annotations overlaid on the images to the user on a display at (408).

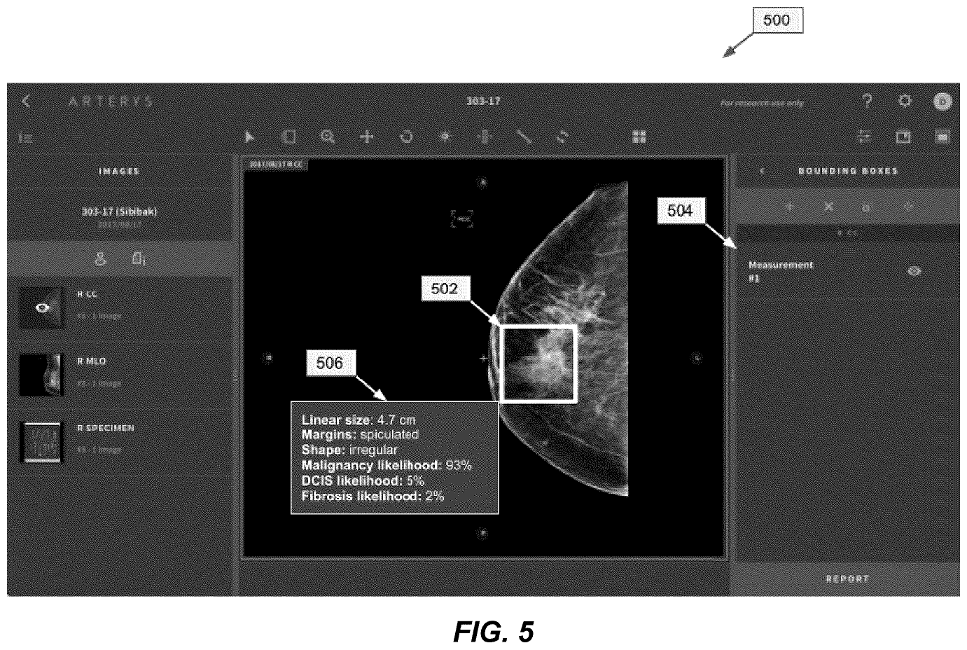

FIG. 5 shows the interface (500) for one embodiment of the Abnormality Detection User Interface as implemented for screening mammography. In this embodiment, an abnormality is indicated with a rectangle at (502). Although a rectangular shape is shown in this embodiment, any geometric shape, such as a triangle or circle, could also be used. A list of detected abnormalities or other annotations is shown in a sidebar at (504). Characteristics related to the abnormality are shown in an overlay at (506). These characteristics may have been determined in any of various ways, including but not limited to:
Manual entry by a user
Collection from a separate database
Calculation by one or more machine learning models,
Calculation by one or more CNN models,
Any combination of the above

In this embodiment, the characteristics are shown adjacent to the annotation overlaid on the pixel data; however, they could also be shown in the sidebar at (504), in a modal dialog, or in other formats. The characteristics may be displayed when the image is first opened, or they may be revealed upon some interaction with the annotation or the sidebar list, such as via a tap or click.

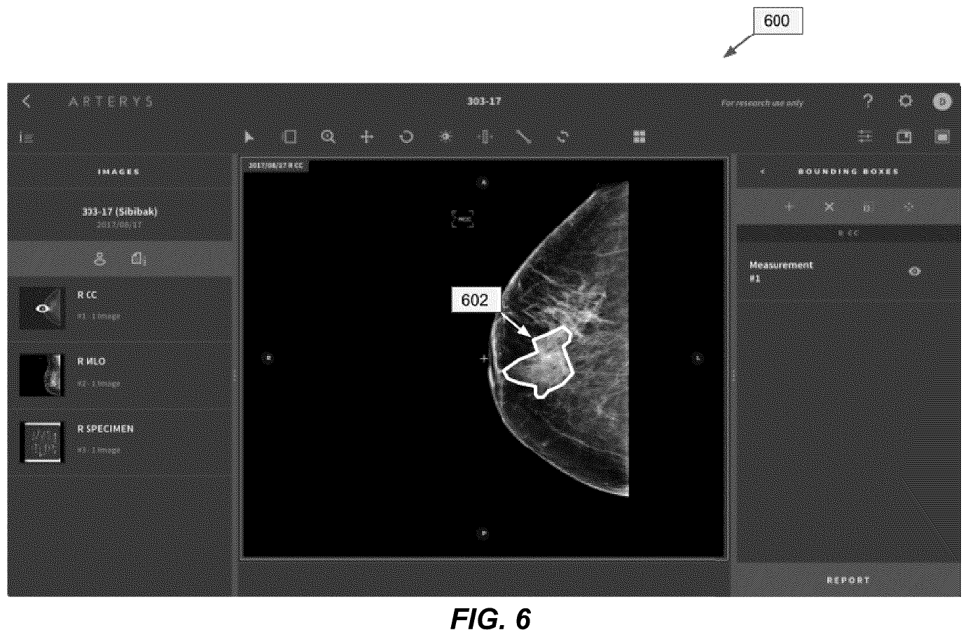

FIG. 6 shows the interface (600) for an alternate embodiment of the Abnormality Detection User Interface wherein the abnormality is indicated with a contour at 602. The contour may be a polygon, spline, or any other kind of regular- or irregular-shaped contour.

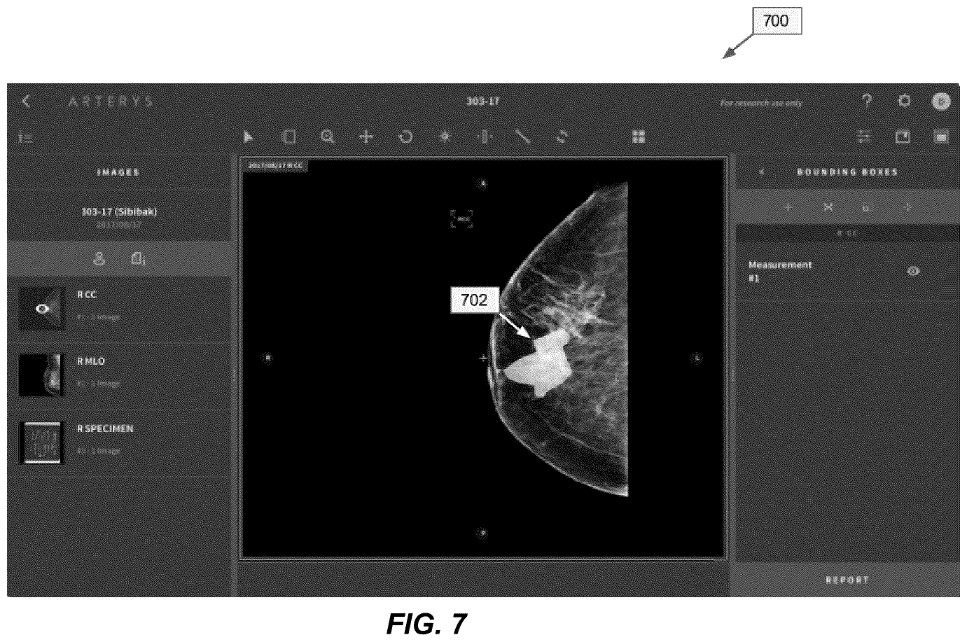

FIG. 7 shows the interface (700) for an alternate embodiment of the Abnormality Detection User Interface wherein the abnormality is indicated with a segmentation mask overlaid on the image at 702. The mask may be opaque or partially translucent. The edges may or may not be highlighted. The contour that defines the edges may be a polygon, spline, or any other kind of regular- or irregular-shaped contour.

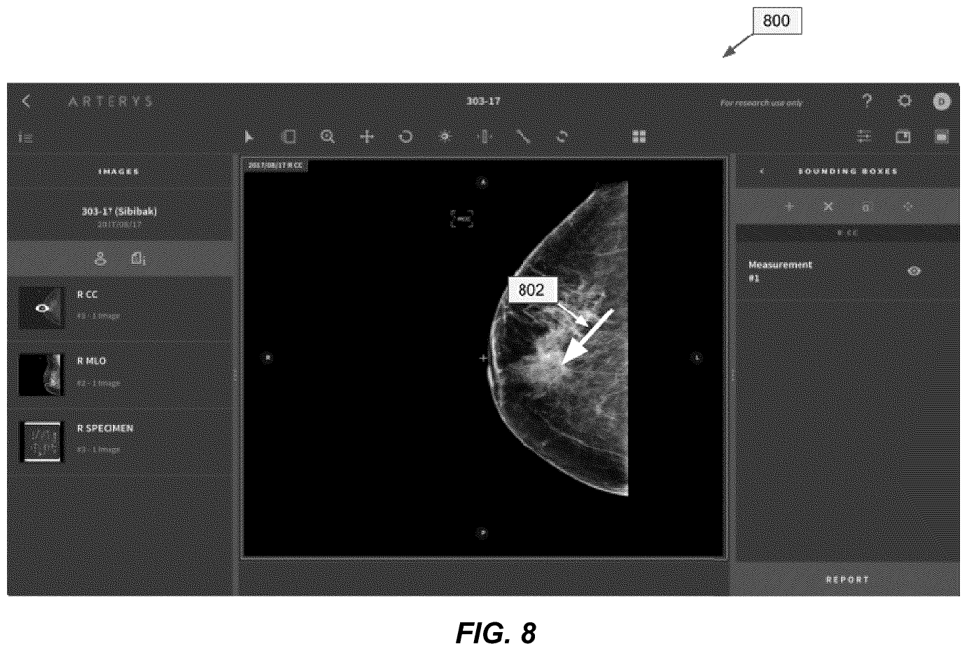

FIG. 8 shows the interface (800) for an alternate embodiment of the Abnormality Detection User Interface wherein the abnormality is indicated with a point indication overlaid on the image at 802. In this embodiment, the indication is an arrow, whose head indicates the point of interest. The point indication may alternately be an overlaid marker, such as a dot or X, it may be any other indicator that signals a specific point on the image, or it may be any combination of these indicators.

### Abnormality Detection Machine Learning Model Inference

The Abnormality Detection Machine Learning Model is a system that ingests image data, possibly in conjunction with other clinical data, and returns an assessment of some subset of abnormality locations, classifications, and probabilities. The embodiments described here operate in the context of mammography screening, but an equivalent system could be used in any medical environment involving an assessment of abnormalities in medical images.

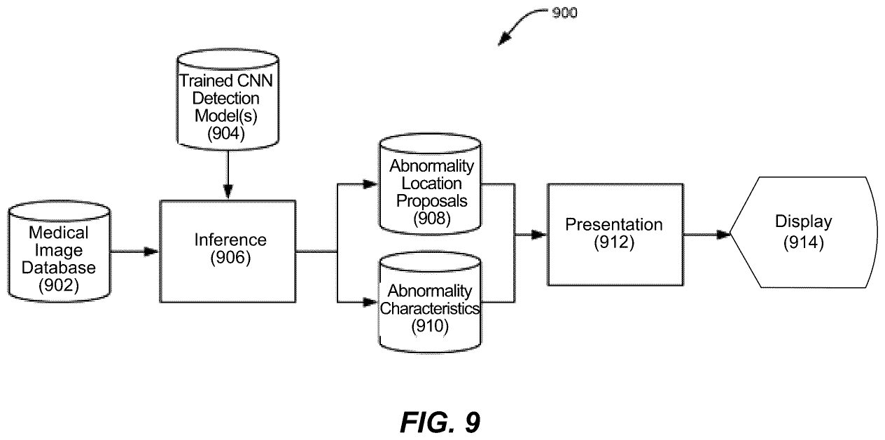

FIG. 9 shows a process flow diagram (900) for one embodiment of an abnormality detection and characterization system. Medical Image Data including one or more medical images, potentially grouped into studies, exists in a Medical Image Database in (902). One or more pre-trained CNN models that are designed to detect and optionally characterize abnormalities exist at (904). The one or more pre-trained CNN models are used to perform inference on at least one medical image at (906). The output of inference includes location proposals (“abnormality location proposals”) for different abnormalities at (908). The abnormality location proposals may take on various forms, including but not limited to:
A rectangular bounding box,
A contour, whose vertices are connected by linear, spline, or other line segments,
A mask of pixels,
One or more individual points (such as the center of mass),
Any combination of the above

The abnormality location proposals may also include associated probabilities for different classes or diagnoses. For example a location proposal may be designated at a malignant lesion with 75% probability, pre-cancerous ductal carcinoma in situ with 20% probability and an intramammary lymph node with 5% probability. The output optionally includes characteristics for those abnormalities at (910). The location proposals may define proposed locations for any abnormalities regardless of subtype, or there may be separate location proposals for specific subclasses of abnormalities (e.g., invasive cancers, non-malignant tumors, cysts, calcifications, etc.). The location proposals (908) may also include confidence indicators or probabilities that the specific proposed location contains the given abnormality. Abnormality characteristics (910), if assessed, may include, without being limited to:
Size,
Margin sharpness,
Roundness,
Opacity,
Spiculation,
Calcifications,
Asymmetry,
Architectural distortions,
Heterogeneity,
Any combination of the above

After being calculated, one or both of the location proposals and characteristics are optionally presented to the user at (912) on a display at (914). In at least some embodiments, only abnormalities detected with high confidence from one or more CNNs are shown. In at least some abnormalities, the likelihood of one or more classes of abnormality or characteristics are displayed. In at least some embodiments, one or both of the location proposals and characteristics are saved to a database for later display or analysis.

One or more of the CNNs at (904) may include a backbone (pre-trained) CNN, a classification CNN or a bounding box regression CNN. The backbone CNN, if included, may be based on a classification, segmentation or other CNN. One or more of the CNNs may be trained with any of various loss functions, including but not limited to focal loss. Focal loss is a modification of standard cross entropy loss such that the loss of predictions whose probabilities are close to the true prediction are downweighted such that their values are reduced when compared to cross entropy loss. One or more CNNs may either operate perform inference on a full input image, or on patches extracted from the input image.

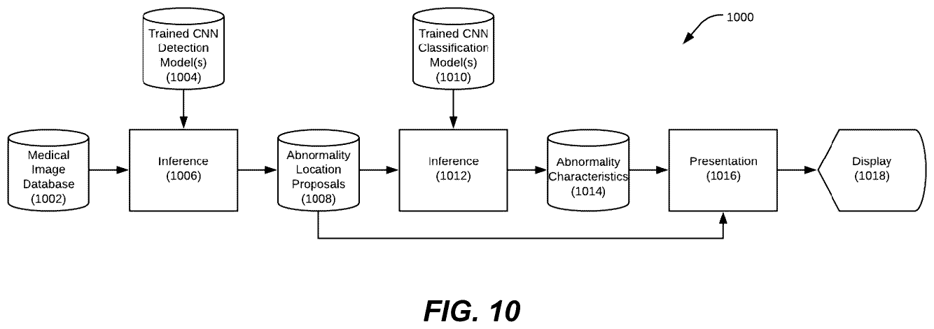

FIG. 10 shows a process flow diagram (1000) for an alternate embodiment of the abnormality detection and characterization system. In this embodiment, there are at least two distinct sets of CNN models, namely one or more detection CNNs at (1004) and one or more classification CNNs at (1010). The detection CNNs at (1004) are primarily responsible for proposing abnormality locations, but they may also provide some characterization of abnormalities for which locations are proposed. The classification CNNs (1010) are primarily responsible for characterizing proposed abnormalities. These characteristics may take on the same format as those in (910). After being calculated, one or both of the location proposals and characteristics are presented to the user at (1016) on a display at (1018).

The detection CNNs at (1004) may have any of the same properties as the CNNs at (904).

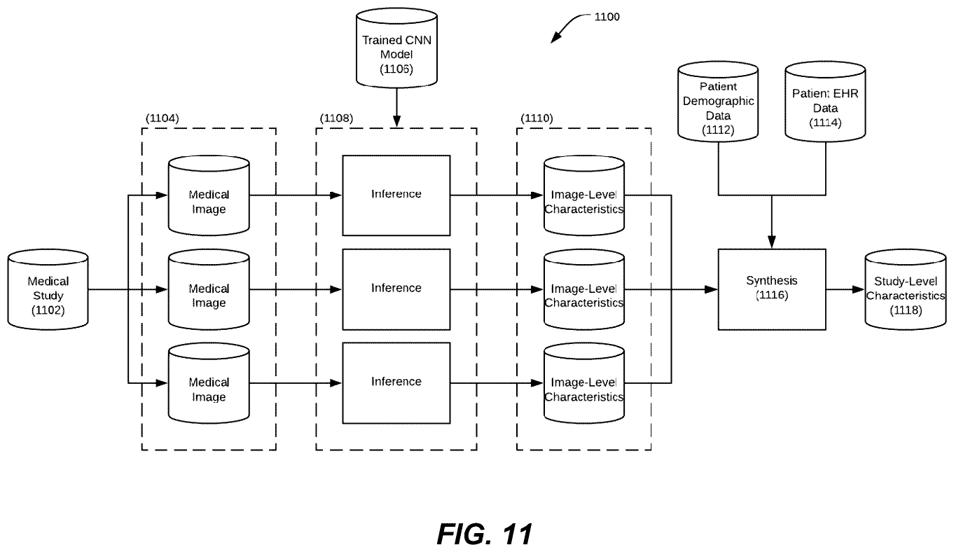

FIG. 11 describes one embodiment of a system (1100) that can be used to characterize a study, including one or more images, based on possibly independent characterizations of its constituent images.

A medical study is loaded at (1102) and is divided into one or more of its constituent medical images at (1104). Note that although a pipeline consisting of three separate images is shown in (1104) through (1110), any number of images could be analyzed in this pipeline. A trained CNN model at (1106) performs inference on each of the images at (1108). Inference may be performed on each image independently, or inference may be performed on some subsets of images simultaneously (for example, multiple images that constitute a volume, or images representing the same anatomy that have been acquired with MRI different pulse sequences). In at least some embodiments, inference includes one or both of detection or characterization of abnormalities. The output of inference is a set of image-level characteristics at (1110). As with inference, these characteristics may be associated with an individual image, or with a collection of images. These characteristics are then synthesized together at (1116), optionally combined with patient demographic data, such as age, sex, lifestyle choices, family disease history, etc., at (1112) or patient electronic health record (EHR) data, such as disease history, test results, procedures, etc. (1114). The output is a set of study level characteristics at (1118).

In at least one embodiment of this system, a study includes mammography screening images that are taken with different views of the two breasts. For example, each of the left and right breasts may have images acquired in the craniocaudal and mediolateral oblique views, resulting in a total of four images. In this embodiment, a lesion detection CNN is applied independently to each image and generates location proposals for detected lesions, along with confidence levels of the proposals for each of various classes of abnormality, such as malignancies and other lesions. A gradient boosted tree algorithm takes in a table containing the list of proposals, their confidence levels, the view and breast side with which the proposals are associated, as well as demographic and clinical data that is associated with breast cancer risk such as age, family history and BRCA mutation status. That gradient boosted tree algorithm then assigns an overall confidence level that any lesion is present in the study. That confidence level may be a continuous score, or it may be quantized to two or more levels of confidence. Quantization to more than 10 classes of likelihood is unlikely to provide significant value over a continuous confidence level.

### Example Processor-Based Device

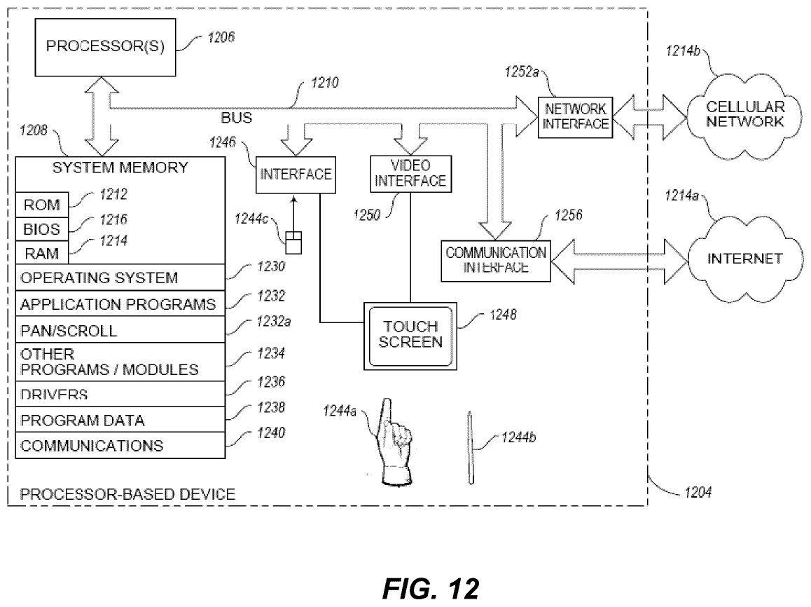

FIG. 12 shows a processor-based device 1204 suitable for implementing the various functionality described herein. Although not required, some portion of the implementations will be described in the general context of processor-executable instructions or logic, such as program application modules, objects, or macros being executed by one or more processors. Those skilled in the relevant art will appreciate that the described implementations, as well as other implementations, can be practiced with various processor-based system configurations, including handheld devices, such as smartphones and tablet computers, wearable devices, multiprocessor systems, microprocessor-based or programmable consumer electronics, personal computers (“PCs”), network PCs, minicomputers, mainframe computers, and the like.

The processor-based device 1204 may include one or more processors 1206, a system memory 1208 and a system bus 1210 that couples various system components including the system memory 1208 to the processor(s) 1206. The processor-based device 1204 will at times be referred to in the singular herein, but this is not intended to limit the implementations to a single system, since in certain implementations, there will be more than one system or other networked computing device involved. Non-limiting examples of commercially available systems include, but are not limited to, ARM processors from a variety of manufactures, Core microprocessors from Intel Corporation, U.S.A., PowerPC microprocessor from IBM, Sparc microprocessors from Sun Microsystems, Inc., PA-RISC series microprocessors from Hewlett-Packard Company, 68xxx series microprocessors from Motorola Corporation.

The processor(s) 1206 may be any logic processing unit, such as one or more central processing units (CPUs), microprocessors, digital signal processors (DSPs), application-specific integrated circuits (ASICs), field programmable gate arrays (FPGAs), etc. Unless described otherwise, the construction and operation of the various blocks shown in FIG. 12 are of conventional design. As a result, such blocks need not be described in further detail herein, as they will be understood by those skilled in the relevant art.

The system bus 1210 can employ any known bus structures or architectures, including a memory bus with memory controller, a peripheral bus, and a local bus. The system memory 1208 includes read-only memory (“ROM”) 1012 and random access memory (“RAM”) 1214. A basic input/output system (“BIOS”) 1216, which can form part of the ROM 1212, contains basic routines that help transfer information between elements within processor-based device 1204, such as during start-up. Some implementations may employ separate buses for data, instructions and power.

The processor-based device 1204 may also include one or more solid state memories, for instance Flash memory or solid state drive (SSD), which provides nonvolatile storage of computer-readable instructions, data structures, program modules and other data for the processor-based device 1204. Although not depicted, the processor-based device 1204 can employ other nontransitory computer- or processor-readable media, for example a hard disk drive, an optical disk drive, or memory card media drive.

Program modules can be stored in the system memory 1208, such as an operating system 1230, one or more application programs 1232, other programs or modules 1234, drivers 1236 and program data 1238.

The application programs 1232 may, for example, include panning/scrolling 1232a. Such panning/scrolling logic may include, but is not limited to logic that determines when and/or where a pointer (e.g., finger, stylus, cursor) enters a user interface element that includes a region having a central portion and at least one margin. Such panning/scrolling logic may include, but is not limited to logic that determines a direction and a rate at which at least one element of the user interface element should appear to move, and causes updating of a display to cause the at least one element to appear to move in the determined direction at the determined rate. The panning/scrolling logic 1232a may, for example, be stored as one or more executable instructions. The panning/scrolling logic 1232a may include processor and/or machine executable logic or instructions to generate user interface objects using data that characterizes movement of a pointer, for example data from a touch-sensitive display or from a computer mouse or trackball, or other user interface device.

The system memory 1208 may also include communications programs 1240, for example a server and/or a Web client or browser for permitting the processor-based device 1204 to access and exchange data with other systems such as user computing systems, Web sites on the Internet, corporate intranets, or other networks as described below. The communications programs 1240 in the depicted implementation is markup language based, such as Hypertext Markup Language (HTML), Extensible Markup Language (XML) or Wireless Markup Language (WML), and operates with markup languages that use syntactically delimited characters added to the data of a document to represent the structure of the document. A number of servers and/or Web clients or browsers are commercially available such as those from Mozilla Corporation of California and Microsoft of Washington.

While shown in FIG. 12 as being stored in the system memory 1208, the operating system 1230, application programs 1232, other programs/modules 1234, drivers 1236, program data 1238 and server and/or browser 1240 can be stored on any other of a large variety of nontransitory processor-readable media (e.g., hard disk drive, optical disk drive, SSD and/or flash memory).

A user can enter commands and information via a pointer, for example through input devices such as a touch screen 1248 via a finger 1244a, stylus 1244b, or via a computer mouse or trackball 1244c which controls a cursor. Other input devices can include a microphone, joystick, game pad, tablet, scanner, biometric scanning device, etc. These and other input devices (i.e., “I/O devices”) are connected to the processor(s) 1206 through an interface 1246 such as touch-screen controller and/or a universal serial bus (“USB”) interface that couples user input to the system bus 1210, although other interfaces such as a parallel port, a game port or a wireless interface or a serial port may be used. The touch screen 1248 can be coupled to the system bus 1210 via a video interface 1250, such as a video adapter to receive image data or image information for display via the touch screen 1248. Although not shown, the processor-based device 1204 can include other output devices, such as speakers, vibrator, haptic actuator, etc.

The processor-based device 1204 may operate in a networked environment using one or more of the logical connections to communicate with one or more remote computers, servers and/or devices via one or more communications channels, for example, one or more networks 1214a, 1214b. These logical connections may facilitate any known method of permitting computers to communicate, such as through one or more LANs and/or WANs, such as the Internet, and/or cellular communications networks. Such networking environments are well known in wired and wireless enterprise-wide computer networks, intranets, extranets, the Internet, and other types of communication networks including telecommunications networks, cellular networks, paging networks, and other mobile networks.

When used in a networking environment, the processor-based device 1204 may include one or more wired or wireless communications interfaces 1252a, 1256 (e.g., cellular radios, WI-FI radios, Bluetooth radios) for establishing communications over the network, for instance the Internet 1214a or cellular network 1214b.

In a networked environment, program modules, application programs, or data, or portions thereof, can be stored in a server computing system (not shown). Those skilled in the relevant art will recognize that the network connections shown in FIG. 12 are only some examples of ways of establishing communications between computers, and other connections may be used, including wirelessly.

For convenience, the processor(s) 1206, system memory 1208, network and communications interfaces 1252a, 1256 are illustrated as communicably coupled to each other via the system bus 1210, thereby providing connectivity between the above-described components. In alternative implementations of the processor-based device 1204, the above-described components may be communicably coupled in a different manner than illustrated in FIG. 12. For example, one or more of the above-described components may be directly coupled to other components, or may be coupled to each other, via intermediary components (not shown). In some implementations, system bus 1210 is omitted and the components are coupled directly to each other using suitable connections.

The foregoing detailed description has set forth various implementations of the devices and/or processes via the use of block diagrams, schematics, and examples. Insofar as such block diagrams, schematics, and examples contain one or more functions and/or operations, it will be understood by those skilled in the art that each function and/or operation within such block diagrams, flowcharts, or examples can be implemented, individually and/or collectively, by a wide range of hardware, software, firmware, or virtually any combination thereof. In one implementation, the present subject matter may be implemented via Application Specific Integrated Circuits (ASICs). However, those skilled in the art will recognize that the implementations disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

Those of skill in the art will recognize that many of the methods or algorithms set out herein may employ additional acts, may omit some acts, and/or may execute acts in a different order than specified.

In addition, those skilled in the art will appreciate that the mechanisms taught herein are capable of being distributed as a program product in a variety of forms, and that an illustrative implementation applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory.

The various implementations described above can be combined to provide further implementations. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to U.S. Provisional Patent Application No. 61/571,908 filed Jul. 7, 2011; U.S. Pat. No. 9,513,357 issued Dec. 6, 2016; U.S. patent application Ser. No. 15/363,683 filed Nov. 29, 2016; U.S. Provisional Patent Application No. 61/928,702 filed Jan. 17, 2014; U.S. patent application Ser. No. 15/112,130 filed Jul. 15, 2016; U.S. Provisional Patent Application No. 62/260,565 filed Nov. 20, 2015; 62/415,203 filed Oct. 31, 2016; U.S. patent application Ser. No. 15/779,445 filed May 25, 2018, U.S. patent application Ser. No. 15/779,447 filed May 25, 2018, U.S. Provisional Patent Application No. 62/415,666 filed Nov. 1, 2016; U.S. patent application Ser. No. 15/779,448, filed May 25, 2018, U.S. Provisional Patent Application No. 62/451,482 filed Jan. 27, 2017; International Patent Application No. PCT/US2018/015222 filed Jan. 25, 2018, U.S. Provisional Patent Application No. 62/501,613 filed May 4, 2017; International Patent Application No. PCT/US2018/030,963 filed May 3, 2018, U.S. Provisional Patent Application No. 62/512,610 filed May 30, 2017; U.S. patent application Ser. No. 15/879,732 filed Jan. 25, 2018; U.S. patent application Ser. No. 15/879,742 filed Jan. 25, 2018; U.S. Provisional Patent Application No. 62/589,825 filed Nov. 22, 2017; U.S. Provisional Patent Application No. 62/589,805 filed Nov. 22, 2017; U.S. Provisional Patent Application No. 62/589,772 filed Nov. 22, 2017; U.S. Provisional Patent Application No. 62/589,872 filed Nov. 22, 2017; U.S. Provisional Patent Application No. 62/589,876 filed Nov. 22, 2017; U.S. Provisional Patent Application No. 62/589,766 filed Nov. 22, 2017; U.S. Provisional Patent Application No. 62/589,833 filed Nov. 22, 2017 and U.S. Provisional Patent Application No. 62/589,838 filed Nov. 22, 2017 are incorporated herein by reference, in their entirety. Aspects of the implementations can be modified, if necessary, to employ systems, circuits and concepts of the various patents, applications and publications to provide yet further implementations.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

This application claims the benefit of priority to U.S. Provisional Application No. 62/770,038, filed Nov. 20, 2018, which application is hereby incorporated by reference in its entirety.

1-29. (canceled)

30. A system for displaying findings on a radiological study, comprising:
at least one nontransitory processor-readable storage medium that stores at least one of processor-executable instructions or data; and
at least one processor communicably coupled to the at least one nontransitory processor-readable storage medium, in operation the at least one processor:
presenting, on a display, image data associated with a radiological study;
presenting, on the display, a location indicator for each of one or more known or suspected abnormalities at the respective location thereof; and
presenting, one the display, one or more characteristics of each of the one or more known or suspected abnormalities.

31-34. (canceled)

35. The system of claim 30, wherein the abnormalities are known or suspected cancer.

36. The system of claim 35, wherein the cancer originates in or has metastasized to at least one of the breast, liver, or lungs.

37. (canceled)

38. (canceled)

39. The system of claim 30, wherein the location indicator comprises a rectangular bounding box.

40. The system of claim 39, wherein the at least one processor provides a user interface that allows for user adjustment of the bounding box via any one or more of translation, zooming, dragging individual edges or dragging individual corners.

41. The method of claim 90, wherein the location indicator comprises a segmentation.

42. The method of claim 41, wherein the segmentation is a polygonal contour.

43. The method of claim 41, wherein the segmentation is a spline contour.

44. The method of claim 41, wherein the segmentation is a mask.

45. The method of claim 30, wherein the location indicator comprises a point indication.

46. The method of claim 45, wherein the point indication is a shape.

47. The method of claim 46, wherein the shape is any of an “x”, an asterisk, a circle, a square, or an arrow.

48. The non-transitory computer-readable medium of claim 91, wherein the acts comprise causing a list of the known or suspected abnormalities to be displayed to a user on a display.

49. The non-transitory computer-readable medium of claim 48, wherein the user's interaction with the location indicator is reflected by changes to the associated abnormality in the list.

50. The non-transitory computer-readable medium of claim 48, wherein the user's interaction with the abnormality in the list is reflected by changes to the associated location indicator.

51. The non-transitory computer-readable medium of claim 91, wherein the one or more characteristic are displayed on the image one of adjacent to or overlapping with the location indicator.

52. The non-transitory computer-readable medium of claim 91, wherein the one or more characteristics are displayed separately from the image data.

53. The non-transitory computer-readable medium of claim 91, wherein the one or more characteristics include any one or more of: abnormality size, opacity, morphology, likelihood of malignancy, possible diagnosis or diagnoses, likelihood of any individual diagnosis; or changes to any of abnormality size, opacity, morphology, likelihood of malignancy, possible diagnosis or diagnoses, likelihood of any individual diagnosis compared to a prior exam.

54-89. (canceled)

90. A computer-implemented method, comprising:
causing a display to present image data associated with a radiological study;
causing the display to present a location indicator for each of one or more known or suspected abnormalities at the respective location thereof; and
causing the display to present one or more characteristics of each of the one or more known or suspected abnormalities.

91. A non-transitory computer-readable medium storing contents that, when executed by one or more processors, cause acts to be performed, the acts comprising:
causing a display to present image data associated with a radiological study;
causing the display to present a location indicator for each of one or more known or suspected abnormalities at the respective location thereof; and
causing the display to present one or more characteristics of each of the one or more known or suspected abnormalities.
